(19) United States
(12) Patent Application Publication
(10) Pub. No.: US 2025/0248712 A1
(43) Pub. Date: Aug. 7, 2025

(54) LAYPERSON AUDIOVISUAL TOURNIQUET

(71) Applicants: THE HENRY M. JACKSON FOUNDATION FOR THE ADVANCEMENT OF MILITARY MEDICINE, INC., Bethesda, MD (US); INNOVITAL, LLC, Calverton, MD (US)

(72) Inventors: Jim MURPHY, Newtown Square, PA (US); Curt Steven KOTHERA, Rockville, MD (US); Craig GOOLSBY, Torrance, CA (US)

(21) Appl. No.: 18/857,032

(22) PCT Filed: Apr. 17, 2023
(86) PCT No.: PCT/US2023/018849

§ 371 Date: Oct. 15, 2024

Related U.S. Application Data
(60) Provisional application No. 63/331,380, filed on Apr. 15, 2022.

(51) Int. Cl. A61B 17/132; A61B 17/00; A61B 90/00

(52) U.S. Cl. CPC A61B 17/1325; A61B 90/03; A61B 2017/00128; A61B 2090/031

(57) ABSTRACT

A tourniquet includes: a strap; a housing containing a winding mechanism, a first end of the strap being attached to a spool shaft of the winding mechanism; a buckle assembly attached to the housing; a buckle tongue attached to a second end of the strap for releasably coupling the strap to the buckle assembly; a first portion of the winding mechanism configured to reduce a length of the strap extending outside of the housing; and an instruction portion that is configured to emit one of audible and visual instructions to a user. The emitting of a particular one of the instructions results from a user step being performed with the tourniquet, the rotating handle is initially decoupled from the first portion of the winding mechanism, and rotation of the rotating handle causes the rotating handle to become mechanically coupled to the spool shaft.

### CROSS-REFERENCE TO RELATED APPLICATION

This application claims the benefit of U.S. Provisional Application No. 63/331,380, which was filed on Apr. 15, 2022, the disclosure of which is hereby incorporated by reference herein in its entirety.

### BACKGROUND

The present invention generally relates to first aid devices to control bleeding, and more particularly to a user-friendly tourniquet device for stopping extremity hemorrhage in a patient through blood vessel occlusion.

Trauma is the leading cause of mortality for those under 45 years of age in the United States. Uncontrolled bleeding is a leading cause of those deaths, as trauma victims can bleed to death from severe wounds in minutes. Therefore, immediate hemorrhage control by bystanders prior to the arrival of emergency medical providers may be lifesaving. One type of device used for such hemorrhage control is an emergency tourniquet.

Tourniquet use in the military has been largely responsible for an 85% reduction in casualty deaths from extremity hemorrhage as reported in a 10-year (2001-2011) analysis. Tourniquet use on the battlefield in Afghanistan and Iraq, often by non-medically trained soldiers, has saved an estimated 1,000-2,000 lives. Similar reductions in extremity hemorrhage deaths may be possible in civilian settings, as well. A group of experts has described the public as “immediate responders” and key to providing point-of-injury hemorrhage control. While the public may not have the training necessary to correctly apply a conventional tourniquet or create an improvised tourniquet, recent studies have demonstrated that the public is willing to place tourniquets on victims.

A common type of commercially available tourniquet generally has four components: (1) a strap that is wrapped around the patient's extremity (e.g., arm or leg); (2) fastening means to form the strap into a loop around the extremity, often a buckle or clasp type of component; (3) a tightening mechanism that reduces the circumference of the loop of strap around the limb such that pressure is applied to occlude the bleeding vessel(s) (e.g., windlass rod); and (4) securing means to prevent the tightening mechanism from loosening after the user has completed tightening (e.g., clip or ratchet). Some tourniquet designs may require additional steps such as a) ensuring that all slack is taken out of the strap before starting the tightening process; or b) locking the tightening mechanism so that the strap does not loosen.

Conventional tourniquets are often applied incorrectly, however, especially by the lay public, and even with just-in-time training. For example, the most commonly used tourniquets are windlass-rod type tourniquets in which the tightening mechanism (3) is a windlass bar that must be twisted, but these devices are not intuitive to use, nor do they provide the user any information to facilitate their application (such as an Automated External Defibrillator might do for someone experiencing cardiac arrest). The windlass bar must be twisted to b) lock the tightening mechanism so that the strap does not loosen, but this increases compression which can cause overpressure. There are multiple “fail points” for a conventional windlass rod tourniquet: placing the tourniquet in an improper position on the limb, inadequate initial tightening, inadequate final tightening, failure to secure the windlass rod in place, muscle relaxation, and combination(s) of errors.

Hence, there is a need for a new tourniquet that is intuitive and easy to use, and that provides assistance or cues to the user during the application process, instructing him/her of what to do and when to do it.

### SUMMARY

In accordance with various embodiments of the disclosure, a tourniquet enables simpler and more intuitive use with fewer steps for the user to perform to stop the bleeding of a patient. Exemplary embodiments include a strap for wrapping around a body part, the strap having a first end and a second end, a buckle attached at the first end, and a winding mechanism for winding and unwinding the strap. The winding mechanism includes a spool shaft for winding and unwinding the strap around the spool shaft. The first end of the strap is attached to the spool shaft and the second end to the buckle. The buckle is used to directly or indirectly couple the ends of the strap to form a loop around the body part. Turning the spool shaft adds compression about the body part. Embodiments provide more intuitive and user-friendly designs for these components in addition to providing user instruction and feedback.

Embodiments of the disclosure include a tourniquet for restricting flow of blood in a body part when a plurality of user steps is completed. The tourniquet includes: a strap configured to be wrapped around a portion of a body part, the strap having a first end and a second end; a housing containing a winding mechanism, the first end of the strap being attached to a spool shaft of the winding mechanism; a buckle assembly attached to the housing; a buckle tongue attached to the second end of the strap for releasably coupling the second end of the strap to the buckle assembly; a rotating handle configured to transfer a rotation motion of a user to the spool shaft; a first portion of the winding mechanism configured to reduce a length of the strap extending outside of the housing in response to the rotating handle being rotated; and an instruction portion that is configured to emit one of audible and visual instructions to a user. The emitting of a particular one of the instructions results from a user step being performed with the tourniquet, the rotating handle is initially decoupled from the first portion of the winding mechanism, and rotation of the rotating handle causes the rotating handle to become mechanically coupled to the spool shaft.

In particular embodiments, a first pawl mechanically engages a first feature on the spool shaft to prevent winding of the strap onto the spool shaft and allow unwinding of the strap from the spool shaft; and a second pawl mechanically engages a second feature to prevent unwinding of the strap from the spool shaft and allow winding of the strap onto the spool shaft.

In particular embodiments, the first pawl engages the spool shaft in a first state while the second pawl is disengaged from the second feature.

In particular embodiments, the second pawl engages the second feature not before the first pawl is disengaged from the spool shaft.

In particular embodiments, each of the instructions corresponds to a different user step.

In particular embodiments, coupling the buckle to the buckle assembly prompts one of the instructions to be emitted.

Particular embodiments include a magnet located on the buckle tongue; and a magnetic switch mounted in the buckle assembly, wherein a proximity of the magnet to the magnetic switch prompts the one of the instructions to be emitted.

Particular embodiments include an input shaft that is rotationally coupled to the spool shaft and through which the rotation motion of the user is transferred to the spool shaft.

In particular embodiments, the input shaft is not parallel to the spool shaft in a strap unwinding state, and the input shaft is parallel to the spool shaft in a strap winding state.

Particular embodiments include an opening in a portion of the housing, wherein a delay between when the first pawl becomes disengaged and when second pawl becomes engaged is caused by an end of the input shaft moving within the opening.

Particular embodiments include a torque limiter that couples the rotating handle to the spooling shaft such that torque above a threshold torque applied to the rotating handle causes the rotating handle to rotate without rotating the spool shaft without destroying the torque limiter.

Particular embodiments include an energy storage element configured to reduce the length of the strap extending outside the housing when energy stored in the energy storage element is released.

Particular embodiments include a platform pivotably coupled to the buckle assembly and configured to contact the body part; and an indicator element that moves relative to a force applied to the body part by the platform, the indicator element indicating to the user when to stop rotating the rotating handle.

Particular embodiments include a platform energy storage element, wherein tightening the tourniquet by rotating the rotating handle increases the force applied to the body part, which causes the platform to pivot relative to the buckle assembly and compresses the platform energy storage element.

Embodiments of the disclosure include a tourniquet for restricting flow of blood in a body part when a plurality of user steps is completed. The tourniquet includes: a strap configured to be wrapped around a portion of a body part, the strap having a first end and a second end; a housing containing a winding mechanism, the first end of the strap being attached to a spool shaft of the winding mechanism; a buckle assembly attached to the housing; a buckle tongue attached to the second end of the strap for releasably coupling the second end of the strap to the buckle assembly; a rotating handle configured to transfer a rotation motion of a user to the spool shaft; a first portion of the winding mechanism configured to reduce a length of the strap extending outside of the housing in response to the rotating handle being rotated; a first pawl that mechanically engages a first feature on the spool shaft to prevent winding of the strap onto the spool shaft and allow unwinding of the strap from the spool shaft; and a second pawl that mechanically engages a second feature to prevent unwinding of the strap from the spool shaft and allow winding of the strap onto the spool shaft.

Particular embodiments include an instruction portion that is configured to emit one of audible and visual instructions to a user, wherein the emitting of a particular one of the instructions results from a user step being performed with the tourniquet.

In particular embodiments, the rotating handle is initially decoupled from the first portion of the winding mechanism, and rotation of the rotating handle causes the rotating handle to become mechanically coupled to the spool shaft.

Particular embodiments include a block that retains the buckle tongue in the buckle assembly in an at rest secured position under tension from the strap; and a retainer that requires the buckle tongue to be pressed farther into the buckle assembly than in the at rest secured position in order to remove the buckle from the buckle assembly.

Embodiments of the disclosure include a tourniquet for restricting flow of blood in a body part when a plurality of user steps is completed. The tourniquet includes: a strap configured to be wrapped around a portion of a body part, the strap having a first end and a second end; a winding mechanism, the first end of the strap being attached to a spool shaft of the winding mechanism; a buckle assembly attached to the winding mechanism; a buckle tongue attached to the second end of the strap for releasably coupling the second end of the strap to the buckle assembly; a rotating handle configured to transfer a rotation motion of a user to the spool shaft; a first portion of the winding mechanism configured to reduce a length of the strap extending outside of the housing in response to the rotating handle being rotated; and an instruction portion that is configured to emit one of audible and visual instructions to a user. The instructions comprise a plurality of different instructions, and each of the plurality of instructions corresponds to one of a different one of a plurality of user steps.

Particular embodiments include a first pawl, and a second pawl, the first pawl mechanically engages a first feature on the spool shaft to prevent winding of the strap onto the spool shaft and allow unwinding of the strap from the spool shaft; and the second pawl mechanically engages a second feature to prevent unwinding of the strap from the spool shaft and allow winding of the strap onto the spool shaft.

Various aspects of the tourniquet, as well as other embodiments, objects, features and advantages of this disclosure, will be apparent from the following detailed description of illustrative embodiments thereof, which is to be read in conjunction with the accompanying drawings.

### BRIEF DESCRIPTION OF THE DRAWINGS

FIG. 1 is a side view of an exemplary tourniquet in accordance with various aspects of the disclosure.

FIG. 2 is a perspective view of the tourniquet of FIG. 1.

FIG. 3 is a perspective view of the tourniquet of FIG. 1.

FIG. 4 is a perspective view of the tourniquet of FIG. 1 with the strap extended.

FIG. 5 is a perspective view of the tourniquet of FIG. 1 in position on a limb.

FIG. 6 is a perspective view of the tourniquet of FIG. 1 in position on a limb.

FIG. 7 is a partial top view of a portion of the tourniquet of FIG. 1.

FIG. 8 is a perspective view of a winding mechanism in accordance with various aspects of the disclosure.

FIG. 9 is a side view of a housing of the tourniquet of FIG. 1.

FIG. 10 is a side view of the housing of the tourniquet of FIG. 1.

FIG. 11 is a perspective view of a portion of the tourniquet of FIG. 1 showing the winding mechanism.

FIG. 12 is a perspective view of a portion of the tourniquet of FIG. 1 showing the winding mechanism.

FIG. 13 is a perspective view of a portion of the tourniquet of FIG. 1 showing the winding mechanism.

FIG. 14 is a perspective view of a portion of the tourniquet of FIG. 1 showing the winding mechanism.

FIG. 15 is a perspective view of a portion of the tourniquet of FIG. 1 showing the winding mechanism.

FIG. 16 is a perspective view of a portion of the tourniquet of FIG. 1 showing the winding mechanism.

FIG. 17 is a perspective view of a portion of the tourniquet of FIG. 1 showing the winding mechanism.

FIG. 18 is a perspective view of a portion of the tourniquet of FIG. 1 showing the winding mechanism.

FIG. 19 is a perspective view of an anti-loosening pawl in accordance with embodiments of the disclosure.

FIG. 20 is a perspective view of an anti-tightening pawl in accordance with embodiments of the disclosure.

FIG. 21 is a perspective view a portion of the tourniquet of FIG. 1.

FIG. 22 is a sectional view a portion of the tourniquet of FIG. 1.

FIG. 23 is a is a perspective view a portion of the tourniquet of FIG. 1.

FIG. 24 is a top view a portion of the tourniquet of FIG. 1.

FIG. 25 is a top view of the tourniquet of FIG. 1.

FIG. 26 is a perspective view of a buckle tongue in accordance with embodiments of the disclosure.

### DETAILED DESCRIPTION OF EMBODIMENTS

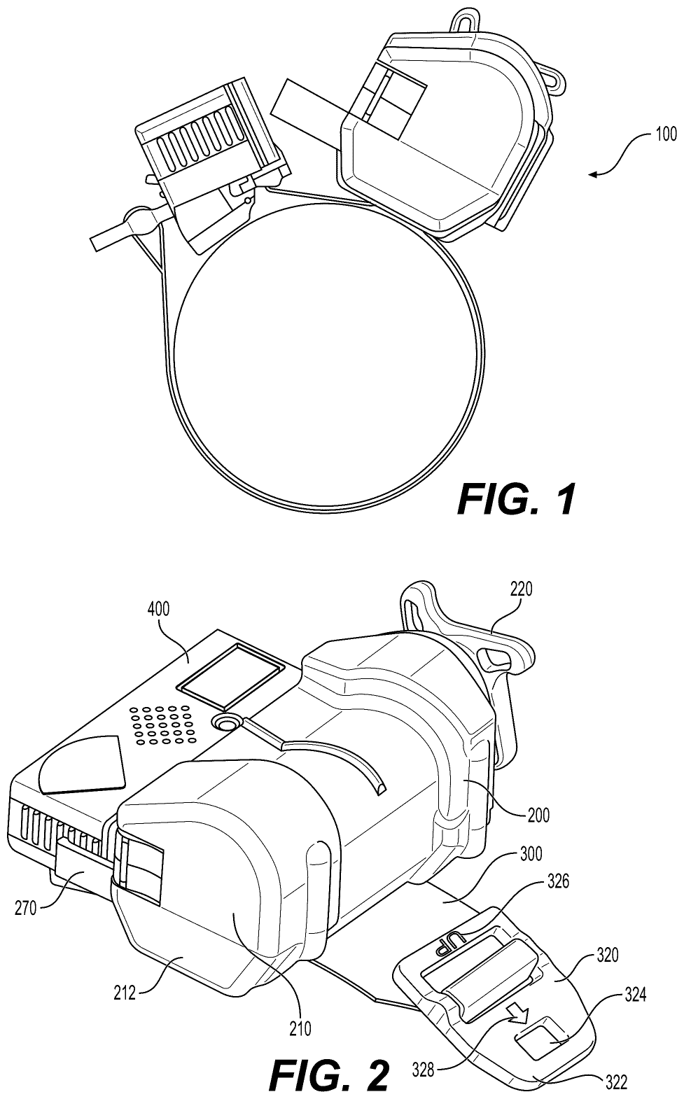

Referring to FIG. 1, in accordance with various embodiments of the disclosure, a device 100 includes a housing 200 containing a spooling shaft 230, a strap 300 wound around the spooling shaft 230 inside of the housing 200, a buckle assembly 400, and a connector strap 500 that connects the buckle assembly 400 to the housing 200. The strap 300 passes through the housing 200 and terminates at a buckle tongue 320. In embodiments, a user directly turns the spooling shaft 230 via a knob 220, or the like, to wind the strap 300 back onto the spooling shaft 230. As the length of the strap 300 around a patient's limb L is reduced, the tension on the strap 300 increases and provides user feedback. Accurate compression is achieved when the tension/torque input required of the device exceeds typical human twisting capabilities, or alternatively triggers a release mechanism or provides another audio/sensory signal.

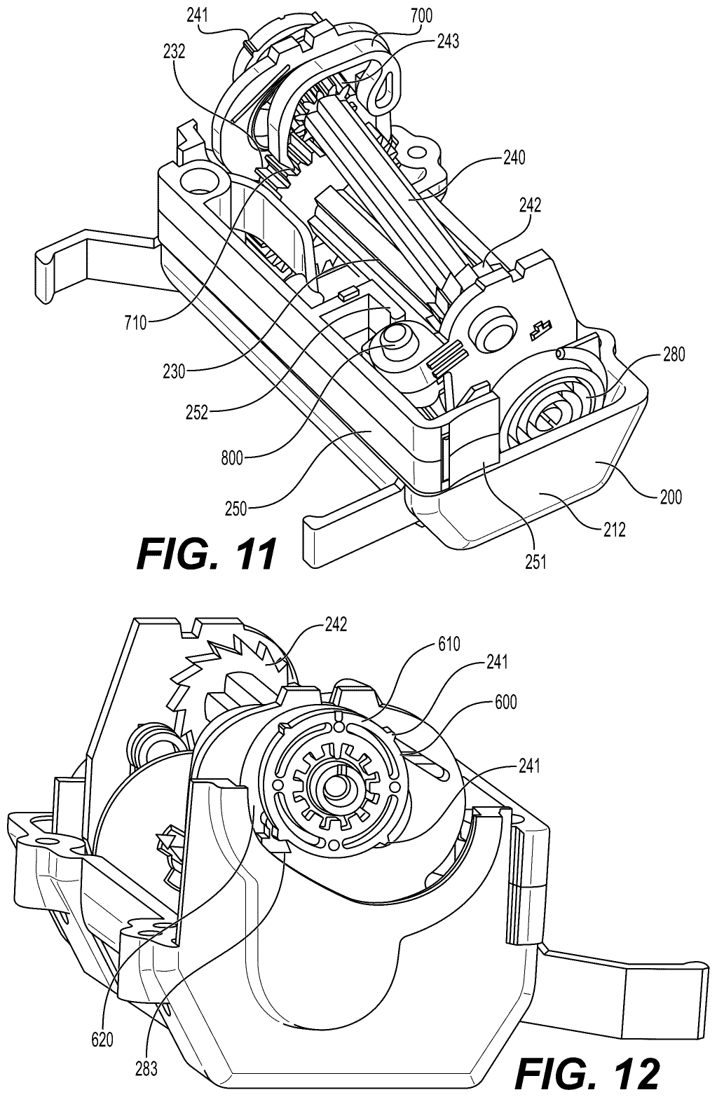

The spooling shaft 230 (FIG. 11) is internal to the housing 200 and acts as a spool about which the strap 300 is wound, both initially (prior to the user pulling it out to wrap it around the patient's limb) and during tightening as the exposed length of the strap 300 decreases to apply circumferential pressure to the limb.

In a first embodiment, the spooling shaft 230 has at least one spooling gear 232, preferably mounted near one of its two ends, and is rotatably situated in the housing 200. In the first embodiment, one end of the spooling shaft 230 is terminated with the twisting knob 220 for user input torque. In this embodiment, the user input torque directly turns the spooling shaft 230 to begin winding the strap 300 back onto the spooling shaft 230 such that the length of strap 300 around the patient's limb is reduced. In embodiments, proper compression is achieved when the tension/torque input required of the device is greater than typical human twisting capabilities and/or triggers a release mechanism or provides another audio/sensory signal.

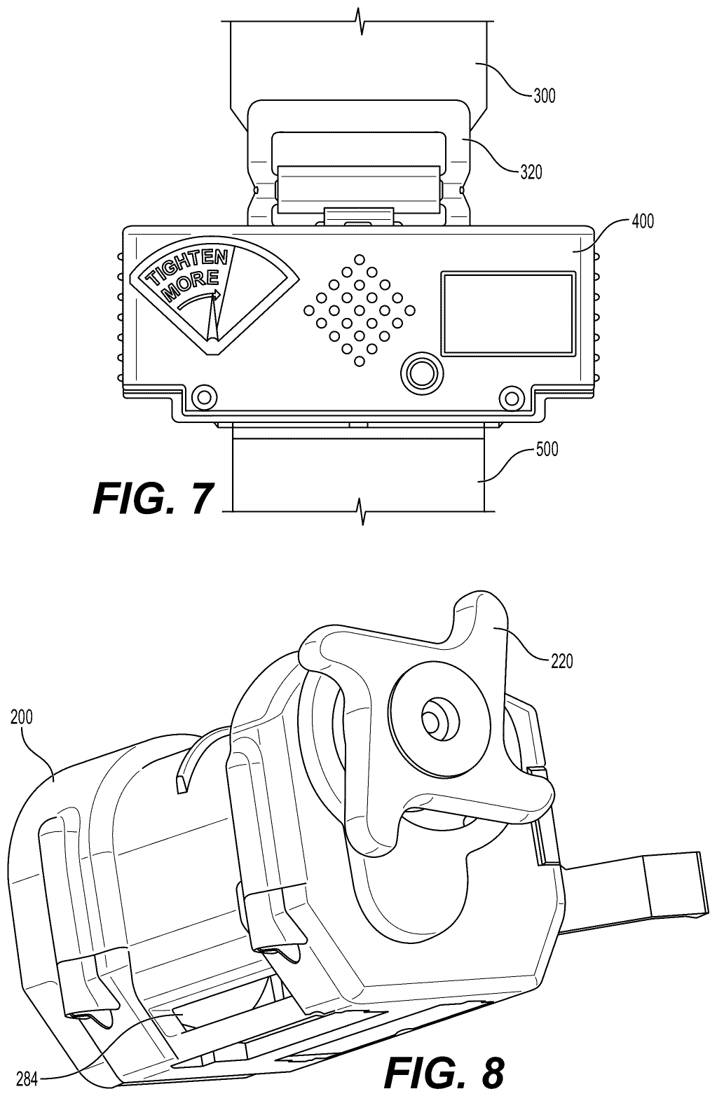

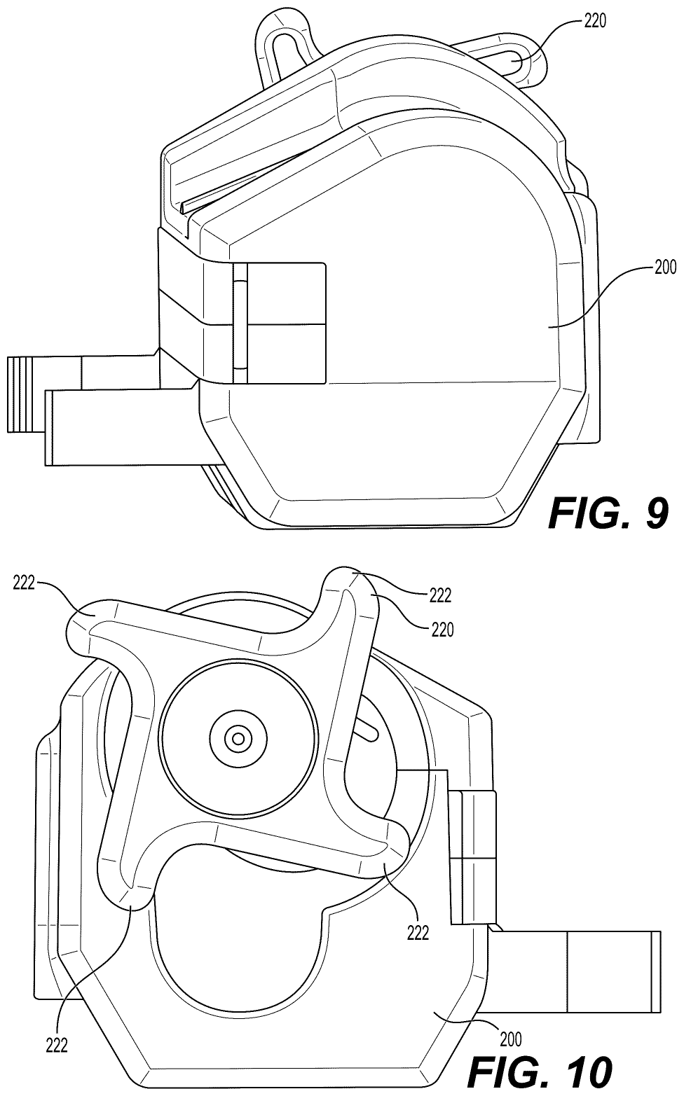

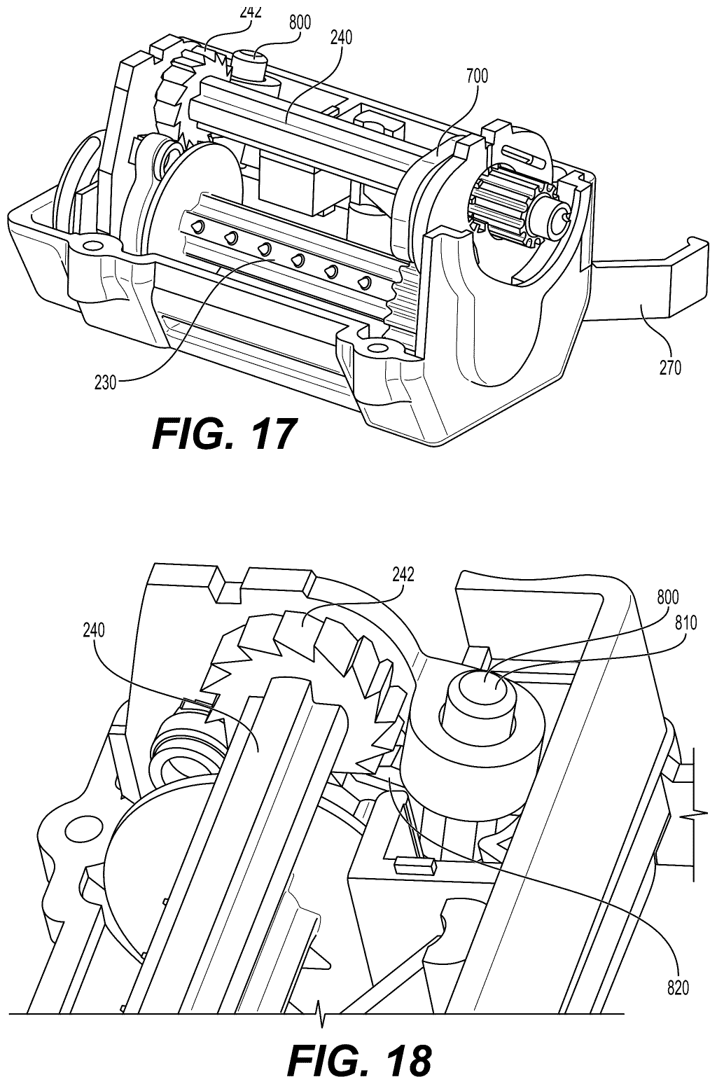

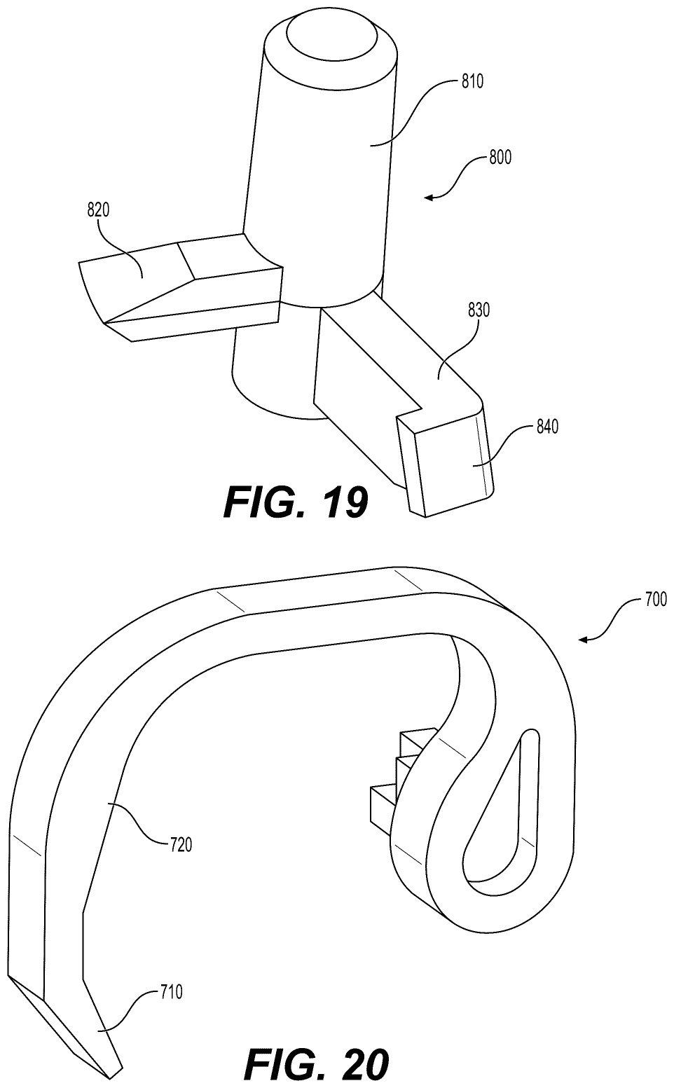

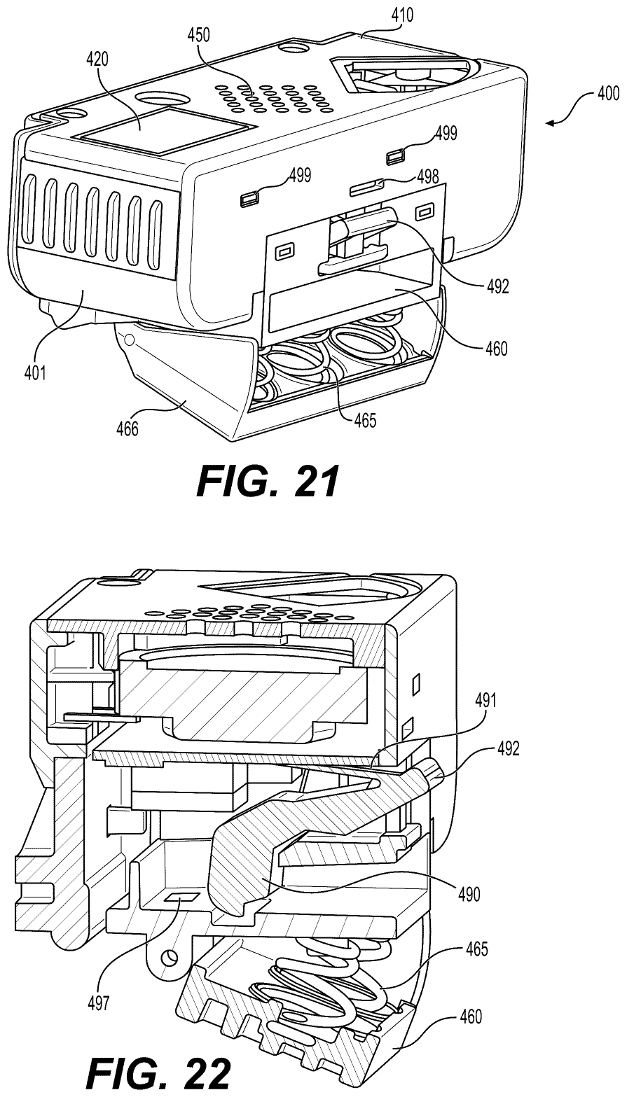

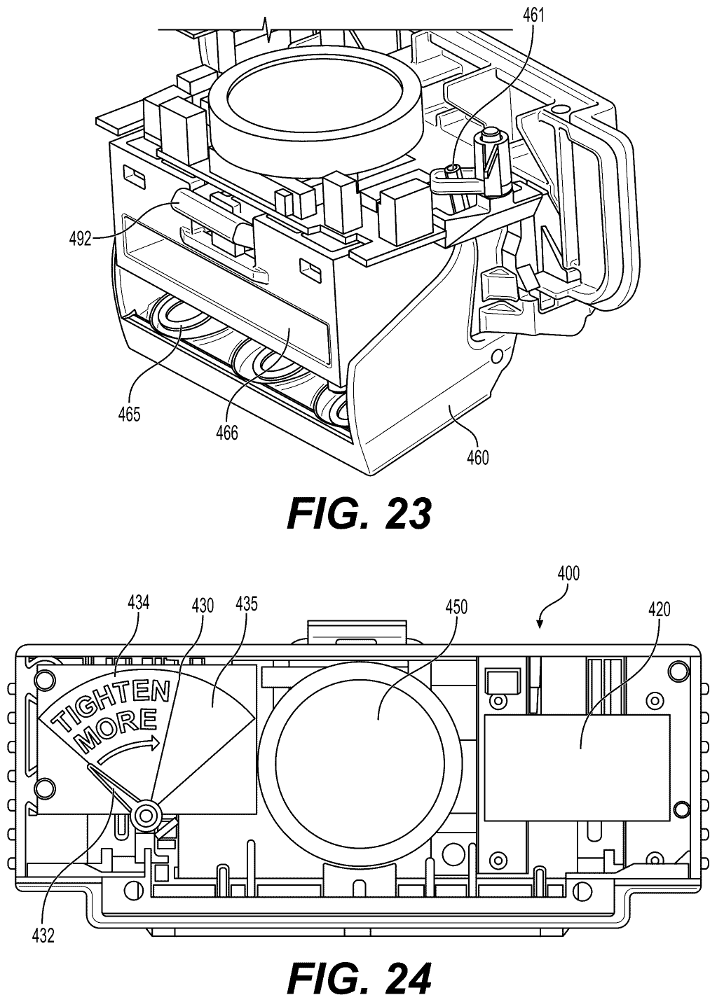

In a second embodiment, shown in FIGS. 1-26, a spooling gear 232 on an end of the spooling shaft 230 is engaged to at least one input gear 243 (generally smaller than the spooling gear 232) mounted on an input shaft 240 that is also rotatably situated in the housing 200 such that the gears 243, 232 of the two shafts 240, 230 are meshed during tightening, and the input shaft 240 is terminated at one end with the twisting knob 220 for user input torque. With the input gear 243 preferably being smaller than the spooling gear 232, mechanical advantage amplifies the human input torque, at the cost of requiring a greater number of turns to wind up the same length of the strap 300. The spooling shaft 230 and the input shaft 240 can be, for example, parallel when tightening, such as for spur gears, or non-parallel, such as for bevel or worm gears. Note that an alternate embodiment has only a spooling shaft (and no separate input shaft), but has a planetary gear system integrated to reduce user input torque required.

The housing 200 includes a base section 212 and a top cover 210. In FIGS. 11-18, top cover 210 has been removed to show parts in the housing 200. As shown in FIG. 10, the twisting knob 220 has a plurality of protrusions 222 that provide better grip of the twisting knob 220. Protrusions 220 are preferably offset in the tightening direction to encourage the user to only turn the knob in the tightening direction (i.e., make it feel less natural to turn the knob in the loosening direction). Indica, such as an arrow pointing in the tightening direction, may also be included on knob 220 to provide a visual cue to the user of how to use the device.

As shown in FIGS. 11 and 16, a winding spring 280 (e.g., clock spring), having two ends, may be attached at one of its ends to one end of the spooling shaft 230 while the other of its ends is fixed relative to the housing 200 (e.g., by the housing 200 or a post), such that relative motion between the spooling shaft 230 and the housing 200 stores potential energy in the winding spring 280. This relative motion specifically is the unwinding of the strap 300 from the spooling shaft 230. That is, as the user pulls out the strap 300 at the beginning of the procedure, the winding spring 280 becomes charged in a manner that will recoil when released, to rewind the strap 300 around the spooling shaft 230 inside the housing 200. This recoiling of the strap 300 can remove excess slack from the strap 300 in the event that the user has pulled out more of the strap 300 than necessary to wrap around the limb. Note that in embodiments of the invention that do not include a winding spring, the user simply winds up the excess slack of the strap 300 by twisting the twisting knob 220 an additional number of turns before pressure is applied to the limb.

In embodiments with the winding spring 280, an anti-tightening mechanism (e.g., ratchet pawl) 700 may be included in the device 100 to prevent release of the winding spring 280 such that the length of the strap 300 pulled out initially by the user will stay out when the user releases it to situate the device 100 on the patient. Otherwise, the strap 300 would recoil back into the device 100 immediately after being released, under the load of the winding spring. In this embodiment, there is also a disengagement mechanism that either switches the anti-tightening mechanism (only allows strap to be unwound) to an anti-loosening mechanism (only allows strap to be wound) or disengages the anti-tightening mechanism when engaging an anti-loosening mechanism (e.g., ratchet pawl) 800 when the user is ready to tighten the tourniquet.

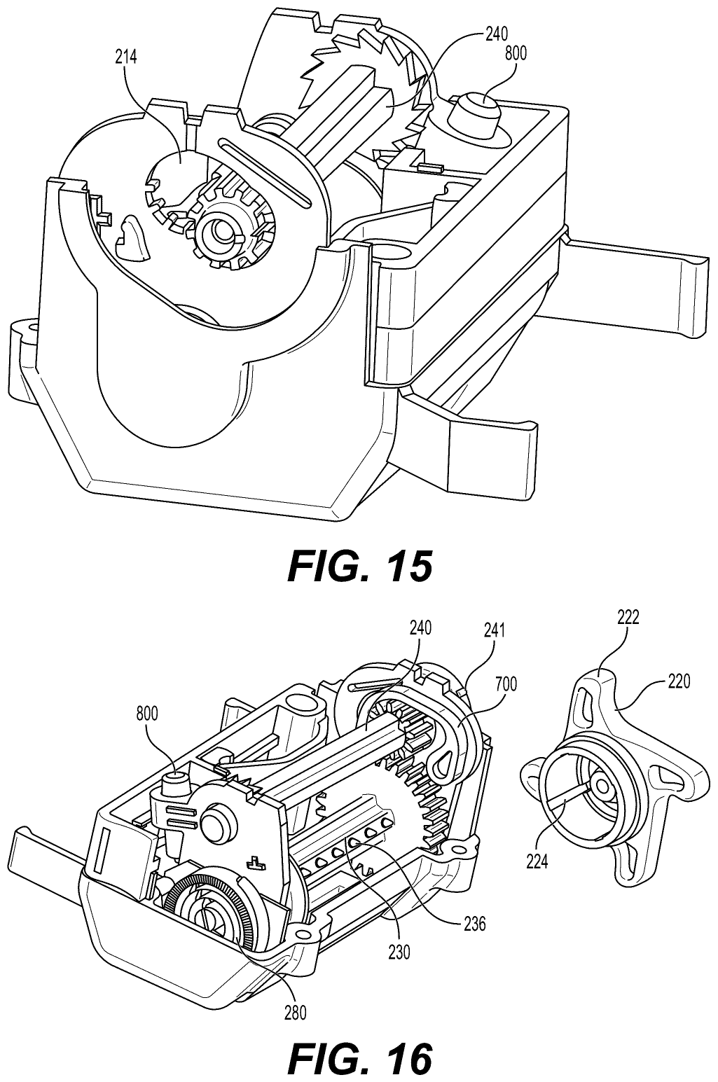

In embodiments, the switching from the anti-tightening mechanism to anti-loosening mechanism occurs without an additional step by the user, but rather as part of one of the general user steps outlined above. This may be achieved by the device having two different ratcheting pawls (or sets thereof) as part of the tightening mechanism: a first pawl (or set of pawls), for example, the anti-tightening pawl 700, to prevent the spool shaft from winding up the strap and only permitting the strap from unwinding therefrom (thereby allowing the effective length of strap to increase); and a second pawl (or set of pawls), for example, the anti-loosening pawl 800, to prevent the spool shaft from unwinding the strap and only permitting the spool shaft to wind the strap (thereby allowing the effective length of strap to decrease). The anti-tightening pawl 700 is engaged with the spool shaft 230 initially, while the anti-loosening pawl 800 is disengaged, in the position shown in FIGS. 11-13. Note that the input shaft 240 and the spooling shaft 230 are not parallel in this position. Through completion of one of the above-outlined user steps, the anti-tightening pawl 700 becomes disengaged from the spool shaft 230 and (possibly after) the anti-loosening pawl 800 becomes engaged with the spool shaft 230. When a winding spring, such as, for example, winding spring 280, is employed, the winding spring 280 can be configured to release its stored mechanical energy as part of this mechanical interaction, thereby recoiling slack in the strap 300. There is preferably a momentary delay between when anti-tightening pawl 700 becomes disengaged and when anti-loosening pawl 800 becomes engaged, during which time strap 300 recoils to remove slack in the strap 300. In one embodiment, this may be implemented in the buckling user step, wherein when the tongue 320 at the end of the strap 300 is inserted into the buckle assembly 400, it moves a finger 710 at an end of the anti-tightening pawl 700 away from teeth on a gear 232 on the spool shaft 230 (see FIG. 11) and moves a finger 820 on the anti-loosening pawl 800 into mechanical interference with a tooth on a gear 242 of the input shaft 240. In embodiments, the finger 710 of the anti-tightening pawl 700 is shaped so that is can flex in one direction to ride over the teeth of the gear 232 on the spooling shaft 230, but engages the teeth on the gear 232 when the gear 232 is rotated in the opposite direction. In embodiments, during the action of turning the twisting knob 220, a gear 243 on the input shaft 240 is moved in an opening 214 from the position shown in FIGS. 11-13 to the position shown in FIG. 15 and presses against an inside surface of the anti-tightening pawl 700, causing the finger 710 of the anti-tightening pawl 700 to disengage the finger 710 from the gear 232. The aforementioned preferred delay in pawl switching may be introduced mechanically via opening 214, where the finger 710 of anti-loosening pawl 700 is disengaged when input shaft 240 begins moving through opening 214, but input gears 243 do not mesh with spool gears 232 and/or finger 820 of anti-loosening pawl 800 does not engage a tooth of gear 242 until turning of knob 220 carries input shaft 240 farther along the path defined by opening 214.

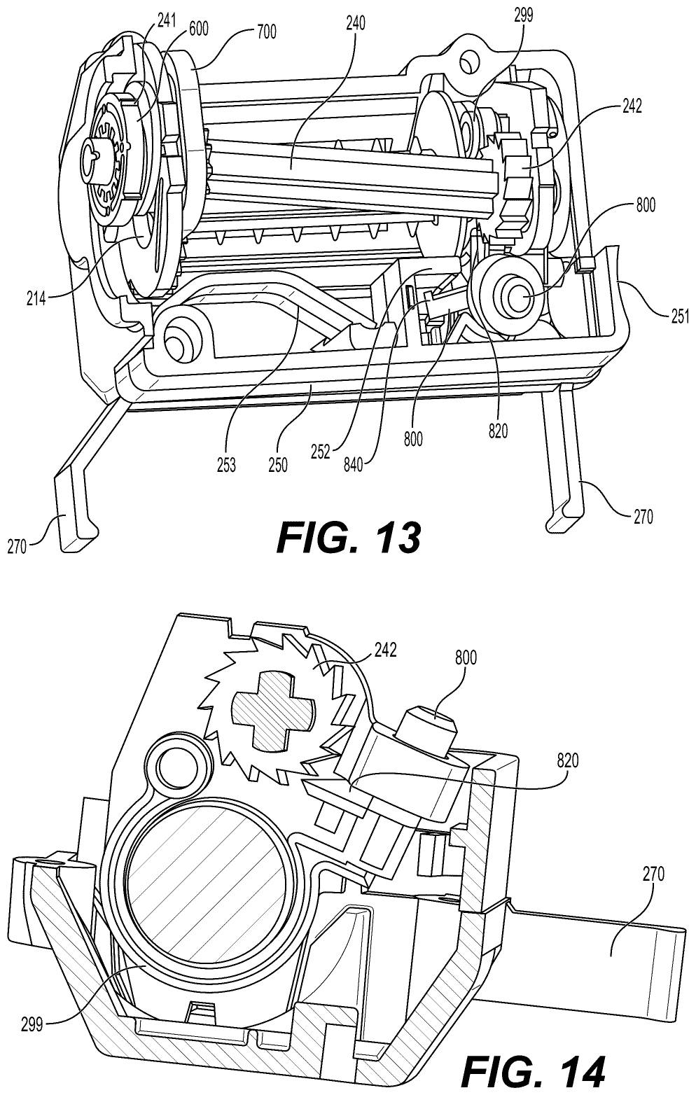

In embodiments, an anti-loosening pawl spring 299 (FIGS. 13 and 14) presses upward on the anti-loosening pawl 800 to urge the finger 820 of the anti-loosening pawl 800 into engagement with the gear 242 of the input shaft 240.

Integrated with the twisting knob 220 may also be a torque limiter that prevents the user from providing too much torque on the shafts that could either overstress the components of the tourniquet device itself or apply too much pressure to the patient's extremity. As shown in FIGS. 12 and 16, a torque limiting cylinder 600 can be provided that mounts externally to the input shaft 240 and that mounts internally to the twisting knob 220, though it could also be an integral part of either within the scope of the invention. The torque limiting cylinder 600 is preferably firmly engaged to the input shaft 240 and flexibly engaged to the twisting knob 220, though the converse could be employed within the same invention. In this embodiment, the torque limiting cylinder 600 is shaped to have flexible segments 610 that keep the twisting knob 220 firmly engaged until the torque between the input shaft 240 and the twisting knob 220 exceeds the design limit, after which the twisting knob 220 (or the input shaft 240) will deform the flexible segments 610 such that the twisting knob 220 effectively slips about the input shaft 240 and does not apply any additional torque or strap tension to the device components, or pressure to the patient's limb. As an example, the torque limiter may be tuned such that internal device components will never see more than a certain fraction of the component's breaking load during tightening. Alternatively, the torque limiter may be tuned to an equivalent pressure (e.g., 500 mmHg) being applied to the patient. Note that the torque limiter is part of some embodiments, but a device with all the other features of the present invention except for a torque limiter still falls within the scope and spirit of the invention.

In embodiments, as shown in FIG. 12, a portion 620 of the torque limiting cylinder 600 engages a boss 283 on the housing 200 or a part fixed to the housing 200 to prevent counter-clockwise (in this example) rotation of the input shaft 240. This physical limit prevents the user from turning the knob 220 in the wrong (i.e., loosening) direction and encourages the user to only turn the knob 220 in the correct (i.e., tightening) direction.

In embodiments, as shown in FIG. 16, a number of recesses 224 are provided on the inside surface of twisting knob 220 that are configured to engage a plurality of protrusions 241 on the torque limiting cylinder 600 that is mounted to the input shaft 240. When an input torque applied to the twisting knob 220 by the user exceeds the threshold torque, the flexible segments 610 of the torque limiting cylinder 600 are pushed inward by the protrusions 241 as the protrusions 241 disengage from the recesses 224, thereby enacting the noted slipping capability to prevent over-pressure.

Shown in FIG. 16 are preferably a plurality of protrusions (for example, barbs) 236 on the spooling shaft 230 that are configured to grip the strap 300, though these are not required.

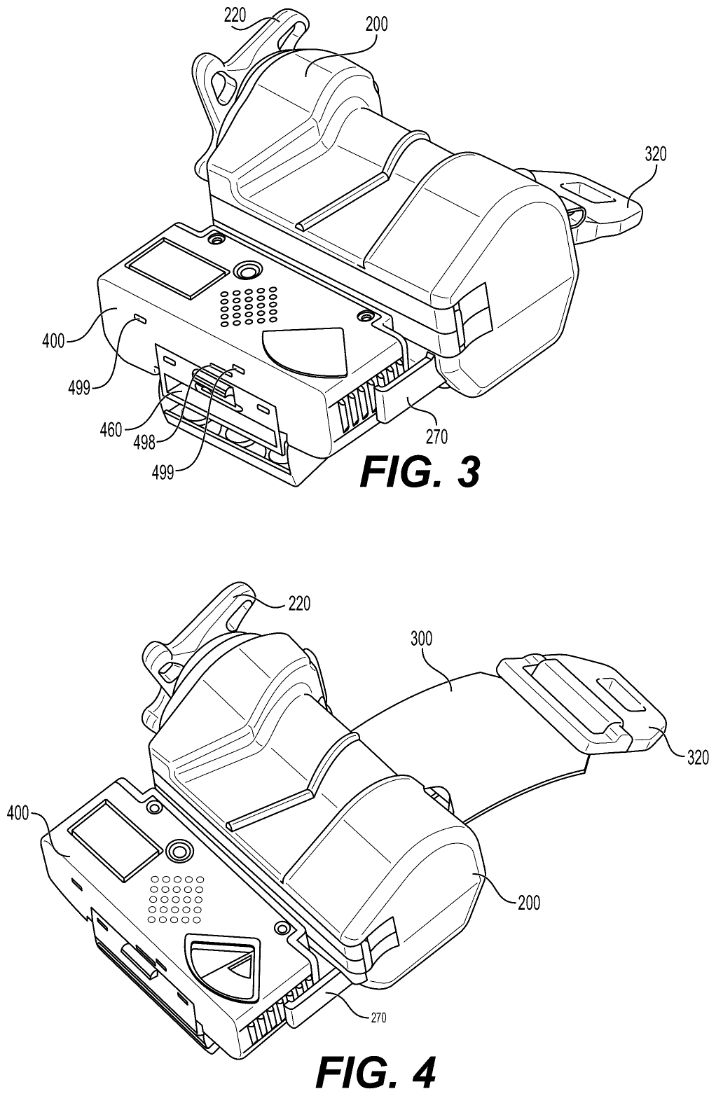

In embodiments where the device 100 has two shafts (i.e., a separate input shaft 240 and spooling shaft 230) that are mechanically engaged, pulling out the strap 300 rotates the twisting knob 220 in the loosening direction. If the user is obstructing the twisting knob 220 (preventing it from rotating) when holding the housing 200, the user may not be able to pull out the strap 300 to wrap it around the patient's limb. The user may not recognize that he or she is obstructing this operation due to the mechanical advantage. To solve this, there exists an embodiment where the input shaft 240 is not mechanically engaged to the spooling shaft 230 when the user first pulls out a length of the strap 300 to wrap around the patient's limb. This effectively de-couples the twisting knob 220 from the spooling shaft 230, so the twisting knob 220 does not turn when the strap 300 is being pulled out initially. This de-coupling feature ensures that the strap 300 can be pulled out in any manner of holding the housing 200 because no external part of the housing 200 moves during this step. In this embodiment, the shafts 230, 240 then become engaged in a separate user step. This separate user step may be a unique step but can be coupled with another step of the tourniquet application process—so as to minimize user steps and failure points. Examples of steps into which this shaft engagement could be coupled within the invention are: (1) insertion of the buckle tongue 320 into a receptacle 460 of the buckle assembly 400 (see FIG. 3); or (2) turning of the twisting knob 220. The former may be accomplished by having the buckle tongue 320 directly or indirectly push the two shafts together such that their gears mesh when the buckle tongue is inserted into the buckle assembly. The latter may be accomplished by having the twisting knob 220 (when turned in the tightening direction) either release stored energy that pushes the two shafts 230, 240 together such that their gears 232, 243 mesh, or direct the input shaft 240 into meshing contact with the spool shaft 230 through a pivoting and/or translation movement. As an example for spur gears, in the former embodiment the two shafts 230, 240 may be parallel or askew at the start before meshing, and in the latter embodiment the two shafts 230, 240 start askew before moving into parallel alignment when meshed. The former embodiment may be applicable to shafts with two sets of gears (e.g., toward both ends, straddling the strap 300), while the latter embodiment is applicable to shafts having a single set of gears (e.g., on one side of the strap 300). Note that this engaging/meshing action activated by the user is also preferably used as the disengagement mechanism to switch from an anti-tightening pawl (such as, for example, anti-tightening pawl 700) to an anti-loosening pawl (such as, for example, anti-loosening pawl 800) such that the tourniquet can be incrementally tightened without losing pressure when the user releases the housing 200 and/or the twisting knob 220. In this sense, the disengagement of the anti-tightening pawl and engagement of the anti-loosening pawl are coupled, synchronized, and automatic with a single user action, as this is preferably coupled to one of the aforementioned user steps for tourniquet application. That is, when the user either inserts the buckle tongue 320 into the buckle assembly 400 or starts twisting the twisting knob 220 (the design may require some nominal amount of rotation before activating, for example), the anti-tightening pawl preferably releases first from engagement, stored energy in the winding spring 280 releases second to begin winding the strap 300, and the anti-loosening pawl 254 engages third to allow continued incremental tightening.

The anti-loosening pawl 800 is an integral part of embodiments of the invention. Certain embodiments of the invention may also include a tension release mechanism 250 (see FIGS. 11 and 13) that releases tension in the strap 300 such that the device can be removed by a clinician or repositioned by a user. The tension release mechanism 250 is preferably user-activated and preferably disengages the anti-loosening pawl 800, at least momentarily, so the strap 300 will loosen and release pressure on the patient. This can be mechanically realized by a user pulling a handle 251 away from the housing 200, which causes an arm 252 to move an end 840 of the anti-loosening pawl 800. Moving the end 840 rotates the anti-loosening pawl 800 and disengages the finger 820 of the anti-loosening pawl 800 from the teeth of the gear 242 on the input shaft 240, thereby releasing the tension. In embodiments, the tension release mechanism 250 is also spring-loaded (by, for example, a coil/torsion spring, a flexible cantilever-like beam 253, etc.) such that it returns to the anti-loosening position after the user releases it. In the example shown, the tension release mechanism 250 is preferably in a location that cannot be easily or inadvertently triggered when operating the device, as this is not a function that a lay user will typically need to use.

In the example shown, the buckle assembly 400 is attached to the housing 200 by the connector strap 500. While a connector strap 500 is preferred, alternative means of joining buckle assembly 400 to housing 200 are allowable within the spirit of the invention. Some such examples include but are not limited to a pivoting attachment, a flex beam attachment, and a rigid connection, preferably stacking the winding spool 230 on top of the buckle assembly 400. Buckle assembly 400 includes a buckle locking mechanism to securely hold/lock the buckle tongue 320 after wrapping the strap 300 around the patient's limb L (i.e., sets the circumference of the loop of the strap 300 around the limb). The buckle locking mechanism can engage the buckle tongue 320 in a linear or rotational manner and can be spring-loaded. Examples of buckle locking mechanisms include, but are not limited to, a block 490 (FIG. 22) that interferes with a cavity or aperture 324 in the buckle tongue 320, teeth or serrations that interfere with similar serrations on the buckle tongue 320, and an angled member that interferes with a cavity 324 and/or tooth-like feature(s) on a forward portion 322 of the buckle tongue 320. That is, the user pulls on the buckle tongue 320 to expose a length of strap 300 that is wrapped around the patient's extremity and then inserts the buckle tongue 320 into the buckle assembly 400 until it clicks and locks in place. In the example shown, the buckle tongue 320 is inserted into the receptacle 460 in the buckle assembly 400.

In embodiments, the buckle locking mechanism is held in an engaged position in its neutral state. When fastening the buckle tongue 320 after wrapping the strap 300 around the limb L, the buckle tongue 320 is inserted into the receptacle 460 in the buckle assembly 400. As the buckle tongue 320 is inserted, the buckle tongue 320 abuts the block 490 and then begins to push it open. In embodiments, the block 490 is spring-loaded (for example, a conventional torsion spring, a flexible bending member 491, etc.) such that continued insertion of the buckle tongue 320 into the buckle assembly 400 increases the stored energy. The stored energy is then released automatically when the buckle tongue 320 is fully inserted and the block 490 snaps into place (e.g., neutral position), catching the feature(s) of the buckle tongue 320 that hold it fixed (i.e., cavity or aperture 324 in the buckle tongue 320). Note that the neutral position may have a pre-load in the lock spring 491 or the snapped/locked position may not be the fully resting state such that the spring still has some nominal loading when engaging the tongue. In embodiments, the block 490 has a release mechanism that allows a medical professional, for example, to fully release the buckle tongue for removal of the device from the patient. In the example shown, the block 490 has a release tab 492 (FIG. 22) that can be pressed (downward in this example) to disengage the block 490 from the cavity 324 of the buckle tongue 320 and allow the buckle tongue 320 to be removed from the buckle assembly 400. Design of tongue cavity 324 and locking block 490 are preferably such that tongue 320 must be pressed farther into receptacle 460 than its resting position under tension. This preferable arrangement makes it very difficult (i.e., practically or physically impossible) for the user to accidentally depress tab 492 and release the device 100 from patient limb L. In this arrangement, tension must first be released from the strap using tension release mechanism 250 before depression of tab 492 can release the tongue 320 from buckle assembly 400.

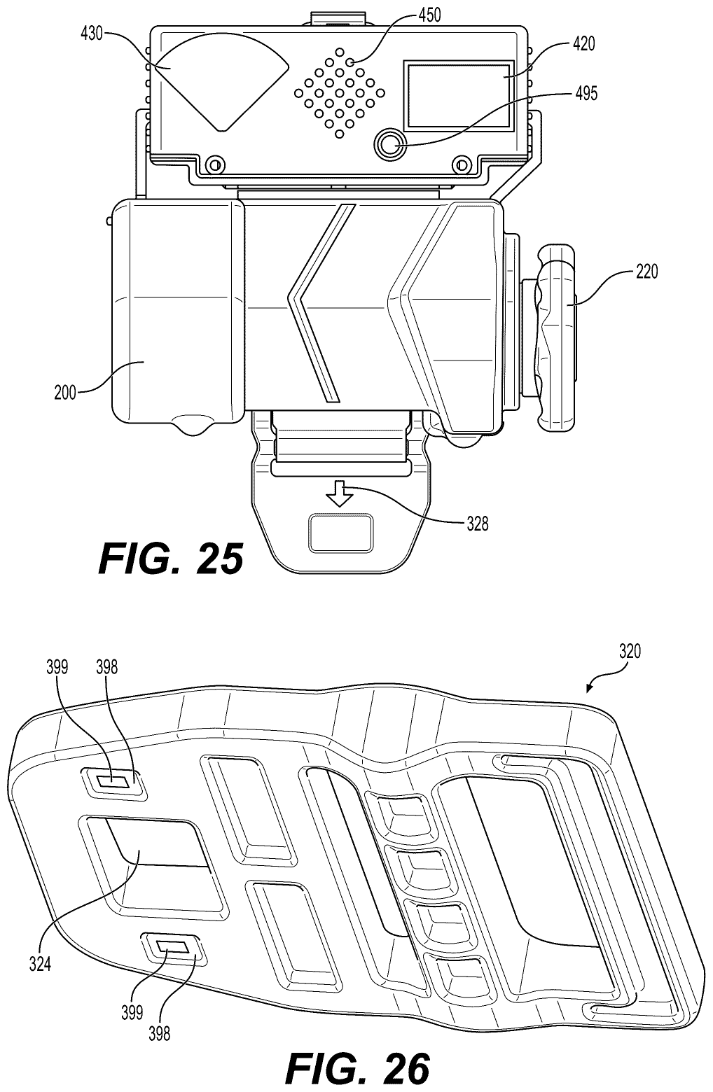

Embodiments include directional indicators such as, for example an arrow, 328 on the buckle tongue 320 to indicate the proper direction of insertion and/or an “UP” indicator 326 on the buckle tongue 320 to indicate the proper orientation (see FIGS. 2 and 25).

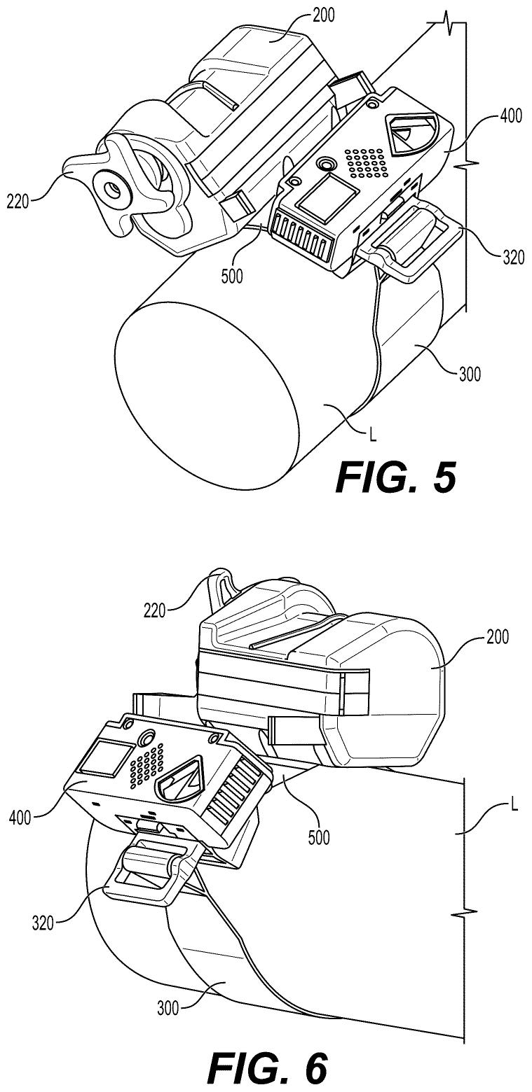

In the example shown, the housing 200 includes two pinching arms 270 that hold the buckle assembly 400 to the housing 200 in an initial position (as in the packaging) as shown in FIGS. 3, 4 and 25. Embodiments include a dock 284 (FIG. 8) for holding a portion of the buckle tongue 320 in the initial position (FIG. 25), though this portion is preferably the rear of the tongue 320 so as not to confuse a user about where to insert the front of tongue 320. These features provide a shipping configuration that has no dangling parts and ensure that when the user removes the device 100 from its packaging, the device 100 comes out as a single user without dangling parts or strap segments. Note that this is true for embodiments where connector strap 500 is a strap segment and not one of the other possible connection types. In the shown embodiment, the user removes device 100 from its package as a single unit, prepares the device for attachment to the patient by pulling the tongue 320 out of its dock 284 along with a length of strap 300 from housing 200, wraps strap 300 around patient limb L, inserts tongue 320 into receptacle 460 of buckle assembly 400 until it clicks into locking place, and then begins to turn twisting knob 220 to tighten the strap. As the tension in strap 300 increases, the buckle assembly 400 will automatically release from pinching arms 270 of housing 200 to better conform to the shape of patient limb L (see FIGS. 5 and 6).

The mechanical components of the invention described above provide an improvement in usability and intuitiveness over existing tourniquets, especially for the untrained public, that help guide the user into correctly applying the tourniquet.

In embodiments, there is no “pre-tightening” step required (both with and without the winding spring), as is required in nearly all existing strap tourniquets on the market. Almost all existing tourniquets have limited tightening ability, meaning that they can only reduce the strap circumference by a fixed length, inherent in their design. As such, pre-tightening is an important first step to ensure that adequate occlusion pressure is applied. If adequate pre-tightening is not applied, occlusion may not be physically possible with the device. This is one of the most common failure modes of existing tourniquets—not applying adequate pressure, either from applying inadequate initial tightening or inadequate final tightening. Embodiments of the disclosure improve upon all these designs by having essentially an unlimited amount of strap length reduction (tightening), as the full length of the strap 300 can be wound back up inside the housing 200.

Audio and visual cues may be employed to aid users in correctly applying tourniquets, particularly, lay users. The embodiment shown provides buckle assembly 400 with a top 410, an audio speaker 450, and a display (for example, an electronic display) 420. The audio speaker 450 and the display 420 give audible and visual cues including, for example, full audio and text instructions of the user steps, static (pictures) or programmable (LCD/OLED displays) graphics to facilitate comprehension, and lighted indicators to draw the user's attention.

One such visual cue provides real-time tightening progress feedback to the user. In embodiments, this is accomplished through the incorporation of a mechanical sensor. Other sensors (electrical, pneumatic, etc.) can also be used, but mechanical sensors are beneficial due to their reliability and simplicity. An exemplary mechanical sensor system includes a spring-loaded mechanism that is kinematically linked to an indicator needle 432 such that deflection of the springs 465 via increased tightening of the strap 300 rotates the needle 432 through an arc that is visible to the user. A progress/feedback graphic 430 behind the needle provides feedback to the user on whether to continue tightening or stop tightening, and can also indicate to the user if the strap has loosened over time. While generally operating like a “bike/tire pump” that is likely familiar to the user, the embodiment shown does not include numbers on the progress graphic 430 in an attempt to reduce cognitive load on the user. The stiffness of the springs 465 in the sensor mechanism and the size of graphical progress zones 434, 435 are then tuned such that the user applies an adequate amount of pressure to most limb sizes. In embodiments, the spring-loaded mechanism includes a base 401, the progress graphic 430 on the base, a platform 466 pivotably coupled to the bottom of the base 401 such that it is in contact with the patient's skin, and a mechanical lever arm 461 extending from the platform 466 to an indicator element (i.e., the indicator needle 432) that moves in relation to the progress graphic 430 when the platform pivots relative to the base as the applied pressure changes.

Some embodiments include one or more audiovisual cue enacted using electronics that drive audiovisual emitters such as the speaker 450 and/or lights 499. The device 100 can be packaged in such a way that power is not connected to the electronics until the user removes the device 100 from its packaging. This may be implemented using a magnetic reed switch in the electronics that is activated by a magnet embedded in the packaging. It may also be implemented using an insulating pull tab that is connected to the packaging on one end and connected to the device 100 on its other end. For example, in embodiments, a slot 498 is provided that receives an end of the pull tab. When the device 100 is removed from its packaging, the pull tab is pulled from the slot 498 and the device 100 is automatically energized. In either of these scenarios, the power is connected to the electronics when the device is removed from the packaging and the device begins its audiovisual sequence without the user having to power the device on. Some embodiments provide both the speaker 450 and the display 420 for redundancy. A first audiovisual instruction set may tell the user to pull out the strap 300 and wrap it around the bleeding limb and then insert the buckle tongue 320 into the buckle assembly 400. Embodiments include blinking lights 499 at the tongue receptacle 460 to direct the user to the appropriate location, and the instructions would make this point clear. In either case, these instructions may be repeated until the step is completed. Once the buckle tongue 320 is fully buckled in place, a sensor or switch 497 can be triggered, which stops the first audiovisual instruction set and begins a second audiovisual instruction set. This sensor switch can be, for example, a magnetic reed switch 497 that reacts to a magnet 399 embedded in a recess 398 in the buckle tongue 320 or an electro-mechanical switch. The second audiovisual instruction may tell the user to twist the twisting knob 220 to tighten the tourniquet until bleeding stops or until the needle 432 enters the “stop tightening” zone 435. In embodiments with indicator lights, the (preferably blinking) light(s) at the tongue insertion receptacle 460 turn off when the second audiovisual instruction set begins and a back-light to the needle display 430 may illuminate.

In embodiments, the second set of instructions repeats a fixed number of times (e.g., three) and then a third instruction set is read aloud and displayed at set intervals and over a longer duration, telling the user to check the patient/tourniquet and to increase tightening if bleeding has resumed or if the needle 432 has fallen out of the “stop tightening” zone 435 as it loosens over time. In embodiments, the second set of instructions repeats until it is completed, as determined by another sensor linked to the device's pressure sensor/gauge needle 432. Then the device 100 advances to the third instruction set as described above, but this third instruction set may differ slightly because the device 100 would be able to determine on its own if the needle 432 has lowered out of the “stop tightening” zone 435.

Embodiments with audio instructions can be provided with a mute button 495 to turn the sound off. The mute button 495 is preferably located in a position that is not easily inadvertently depressed when operating the device 100. The purpose of the mute button 495 is to give the user an option to silence the device 100 if the current situation would benefit.

It is to be understood that this present disclosure is not limited to the specific embodiments and methods described, as specific components and/or conditions may vary. Furthermore, the terminology used herein is used only for the purpose of describing particular embodiments of the present disclosure and is not intended to be limiting in any way.

While multiple exemplary non-limiting embodiments have been presented in the foregoing detailed description, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing the exemplary embodiment or exemplary embodiments. It should be understood that various changes can be made in the function and arrangement of elements without departing from the scope of the disclosure as set forth in the appended claims and the legal equivalents thereof.

Although the illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

Various changes to the foregoing described and shown structures will now be evident to those skilled in the art. Accordingly, the particularly disclosed scope of the invention is set forth in the following claims.

1. A tourniquet for restricting flow of blood in a body part when a plurality of user steps is completed, the tourniquet comprising:
a strap configured to be wrapped around a portion of a body part, the strap having a first end and a second end;
a housing containing a winding mechanism, the first end of the strap being attached to a spool shaft of the winding mechanism;
a buckle assembly attached to the housing;
a buckle tongue attached to the second end of the strap for releasably coupling the second end of the strap to the buckle assembly;
a rotating handle configured to transfer a rotation motion of a user to the spool shaft;
a first portion of the winding mechanism configured to reduce a length of the strap extending outside of the housing in response to the rotating handle being rotated; and
an instruction portion that is configured to emit one of audible and visual instructions to a user,
wherein the emitting of a particular one of the instructions results from a user step being performed with the tourniquet,
the rotating handle is initially decoupled from the first portion of the winding mechanism, and
rotation of the rotating handle causes the rotating handle to become mechanically coupled to the spool shaft.

2. The tourniquet of claim 1, further comprising a first pawl, and a second pawl,
wherein the first pawl mechanically engages a first feature on the spool shaft to prevent winding of the strap onto the spool shaft and allow unwinding of the strap from the spool shaft; and
the second pawl mechanically engages a second feature to prevent unwinding of the strap from the spool shaft and allow winding of the strap onto the spool shaft.

3. The tourniquet of claim 2, wherein the first pawl engages the spool shaft in a first state while the second pawl is disengaged from the second feature.

4. The tourniquet of claim 3, wherein the second pawl engages the second feature not before the first pawl is disengaged from the spool shaft.

5. The tourniquet of claim 1, wherein each of the instructions corresponds to a different user step.

6. The tourniquet of claim 1, wherein coupling the buckle to the buckle assembly prompts one of the instructions to be emitted.

7. The tourniquet of claim 6, further comprising a magnet located on the buckle tongue; and a magnetic switch mounted in the buckle assembly, wherein a proximity of the magnet to the magnetic switch prompts the one of the instructions to be emitted.

8. The tourniquet of claim 1, further comprising an input shaft that is rotationally coupled to the spool shaft and through which the rotation motion of the user is transferred to the spool shaft.

9. The tourniquet of claim 8, wherein the input shaft is not parallel to the spool shaft in a strap unwinding state, and the input shaft is parallel to the spool shaft in a strap winding state.

10. The tourniquet of claim 8, further comprising an opening in a portion of the housing,
wherein a delay between when the first pawl becomes disengaged and when second pawl becomes engaged is caused by an end of the input shaft moving within the opening.

11. The tourniquet of claim 1, further comprising a torque limiter that couples the rotating handle to the spooling shaft such that torque above a threshold torque applied to the rotating handle causes the rotating handle to rotate without rotating the spool shaft without destroying the torque limiter.

12. The tourniquet of claim 1, further comprising an energy storage element configured to reduce the length of the strap extending outside the housing when energy stored in the energy storage element is released.

13. The tourniquet of claim 1, further comprising a platform pivotably coupled to the buckle assembly and configured to contact the body part; and
an indicator element that moves relative to a force applied to the body part by the platform, the indicator element indicating to the user when to stop rotating the rotating handle.

14. The tourniquet of claim 13, further comprising a platform energy storage element,
wherein tightening the tourniquet by rotating the rotating handle increases the force applied to the body part, which causes the platform to pivot relative to the buckle assembly and compresses the platform energy storage element.

15. A tourniquet for restricting flow of blood in a body part when a plurality of user steps is completed, the tourniquet comprising:
a strap configured to be wrapped around a portion of a body part, the strap having a first end and a second end;
a housing containing a winding mechanism, the first end of the strap being attached to a spool shaft of the winding mechanism;
a buckle assembly attached to the housing;
a buckle tongue attached to the second end of the strap for releasably coupling the second end of the strap to the buckle assembly;
a rotating handle configured to transfer a rotation motion of a user to the spool shaft;
a first portion of the winding mechanism configured to reduce a length of the strap extending outside of the housing in response to the rotating handle being rotated;
a first pawl that mechanically engages a first feature on the spool shaft to prevent winding of the strap onto the spool shaft and allow unwinding of the strap from the spool shaft; and
a second pawl that mechanically engages a second feature to prevent unwinding of the strap from the spool shaft and allow winding of the strap onto the spool shaft.

16. The tourniquet of claim 15, further comprising an instruction portion that is configured to emit one of audible and visual instructions to a user,
wherein the emitting of a particular one of the instructions results from a user step being performed with the tourniquet.

17. The tourniquet of claim 15, wherein the rotating handle is initially decoupled from the first portion of the winding mechanism, and
rotation of the rotating handle causes the rotating handle to become mechanically coupled to the spool shaft.

18. The tourniquet of claim 15, further comprising a block that retains the buckle tongue in the buckle assembly in an at rest secured position under tension from the strap; and
a retainer that requires the buckle tongue to be pressed farther into the buckle assembly than in the at rest secured position in order to remove the buckle from the buckle assembly.

19. A tourniquet for restricting flow of blood in a body part when a plurality of user steps is completed, the tourniquet comprising:
a strap configured to be wrapped around a portion of a body part, the strap having a first end and a second end;
a winding mechanism, the first end of the strap being attached to a spool shaft of the winding mechanism;
a buckle assembly attached to the winding mechanism;
a buckle tongue attached to the second end of the strap for releasably coupling the second end of the strap to the buckle assembly;
a rotating handle configured to transfer a rotation motion of a user to the spool shaft;
a first portion of the winding mechanism configured to reduce a length of the strap extending outside of the housing in response to the rotating handle being rotated; and
an instruction portion that is configured to emit one of audible and visual instructions to a user,
wherein the instructions comprise a plurality of different instructions, and
each of the plurality of instructions corresponds to one of a different one of a plurality of user steps.

20. The tourniquet of claim 19, further comprising a first pawl, and a second pawl,
wherein the first pawl mechanically engages a first feature on the spool shaft to prevent winding of the strap onto the spool shaft and allow unwinding of the strap from the spool shaft; and
the second pawl mechanically engages a second feature to prevent unwinding of the strap from the spool shaft and allow winding of the strap onto the spool shaft.
